(19) United States
(12) Patent Application Publication
(10) Pub. No.: US 2003/0050660 A1
(43) Pub. Date: Mar. 13, 2003

(54) Eccentric catheter shaft

(75) Inventor: Steven S. Hackett, Maple Grove, MN (US)

Correspondence Address: Robert E. Atkinson, CROMPTON, SEAGER & TUFTE, LLC, Suite 895, 331 Second Avenue South, Minneapolis, MN, 55401-2246, US

(73) Assignee: SciMed Life Systems, Inc.

(21) Appl. No.: 09/948,517

(22) Filed: Sep. 7, 2001

(51) Int. Cl. A61M 29/00

(52) U.S. Cl. 606/194

(57) ABSTRACT

An intravascular device having a tubular shaft with an outer wall and an inner wall which divides the outer wall into two or more lumens. The shaft also includes one or more regions of modified flexibility extending longitudinally along the outer wall. Absent the regions of modified flexibility, the inner wall would create an imbalance of material and flexibility about the center axis of the shaft. The regions of modified flexibility are positioned to reduce any such imbalance, thereby providing more uniform flexibility. The regions of modified flexibility also provide for more uniform torque transmission, and thereby reduce whipping effects. The regions of modified flexibility may comprise one or more regions of decreased wall thickness in the outer wall, one or more spines extending longitudinally along the outer wall, or a combination thereof.

### FIELD OF THE INVENTION

The present invention generally relates to intravascular medical devices. More specifically, the present invention relates to multi-lumen intravascular medical devices such as balloon catheters.

### BACKGROUND OF THE INVENTION

Intravascular devices are commonly used to diagnose and treat various types of vascular disease. For example, coronary artery disease (CAD) may be treated utilizing a procedure called percutaneous transluminal coronary angioplasty (PTCA). In a typical PTCA procedure, intravascular devices are inserted into the patient's vascular system at a remote access site such as the femoral artery near the groin. The intravascular devices are navigated through the femoral artery and the descending aorta, over the aortic arch, down the ascending aorta, and into the targeted coronary artery.

The path from the remote access site to the targeted coronary artery is established and maintained utilizing a conventional guide catheter and guidewire. The guide catheter extends from a point outside the patient's body, through the remote access site, to the ostium of the targeted coronary artery. The guidewire extends from a point outside the patient's body, through the guide catheter, and across the treatment site of the targeted coronary artery. A balloon catheter may then be advanced over the guidewire through the guide catheter until the distally mounted balloon is positioned across the treatment site. The balloon is then inflated to dilate the vascular restriction, thereby opening the artery and restoring blood flow.

Different types of balloon catheters are suitable for use in this type of procedure. Balloon catheters that are designed for use in combination with a guidewire as discussed above are typically referred to as over-the wire (OTW) or rapid exchange (RX) type balloon catheters. OTW and RX type balloon catheters include an elongate shaft having an inflation lumen and a guidewire lumen. In an OTW type balloon catheter, the guidewire lumen extends from the proximal end of the catheter to the distal end of the catheter. In an RX type balloon catheter, the guidewire lumen extends from a point distal of the proximal end to the distal end of the catheter. In both cases, at least a portion of the elongate shaft includes an inflation lumen and a guidewire lumen.

In typical OTW and RX type balloon catheters, the guidewire lumen and the inflation lumen are defined by either a coaxial shaft structure or a dual lumen shaft structure. In a coaxial design, the elongate shaft includes an inner tube coaxially disposed in an outer tube such that the inner tube defines a circular guidewire lumen and the outer tube defines an annular inflation lumen. An example of a typical coaxial shaft design is disclosed in U.S. Pat. No. 4,323,071 to Simpson et al. In dual lumen shaft designs, a single tubular extrusion is used to define separate guidewire and inflation lumens extending side-by-side. An example of a dual lumen shaft design is disclosed in U.S. Pat. No. 4,782,834 to Maguire et al.

One advantage provided by a coaxial type shaft design, as compared to a dual lumen type shaft design, is uniform flexibility due to the coaxial arrangement of parts. In other words, the coaxial type shaft design has the same flexibility in all planes of flexure, whereas the dual lumen type shaft design has non-uniform flexibility in differenct planes of flexure due to the imbalance of material relative to the longitudinal center axis of the catheter shaft. The non-uniformity in flexibility of the dual lumen type shaft design may compromise trackability and torqueability of the catheter, thereby reducing the ability of the catheter to navigate tortuous vasculature. One advantage provided by a dual lumen type shaft design is reduced frictional loss and less resistance to fluid flow in the inflation lumen as compared to a coaxial type shaft design having the same cross-sectional area. This provides better balloon inflation/deflation rates which are desirable for various clinical reasons.

Accordingly, there is a need for a shaft design for an intravascular device such as a balloon catheter wherein the flexibility is uniform in all planes of flexure and the frictional loss in the inflation lumen is minimized.

### SUMMARY OF THE INVENTION

To address this need, the present invention provides an intravascular device, such as a balloon catheter, having a tubular shaft with an outer wall and an inner wall. The inner wall divides the outer wall into two or more lumens, such as a larger crescent-shaped lumen which may be used as an inflation lumen, and a smaller circle-shaped lumen which may be used as a guidewire lumen. The shaft also includes one or more regions of modified flexibility extending longitudinally along the outer wall. Absent the regions of modified flexibility, the inner wall would create an imbalance of material and flexibility about the center axis of the shaft. The regions of modified flexibility are positioned to reduce any such imbalance, thereby providing more uniform flexibility, without compromising the fluid dynamic capabilities of the lumens. The regions of modified flexibility also provide for more uniform torque transmission, and thereby reduce whipping effects.

In one embodiment, the regions of modified flexibility comprise one or more regions of decreased wall thickness in the outer wall. In another embodiment, the regions of modified flexibility comprise one or more spines extending longitudinally along the outer wall. In yet another embodiment, the regions of modified flexibility comprise a combination of these features.

### BRIEF DESCRIPTION OF THE DRAWINGS

FIG. 1 is a plan view of an intravascular device in accordance with the present invention, shown in the exemplary form of a balloon catheter;

FIG. 2A is a cross-sectional view and FIG. 3A is a partial isometric view of an embodiment of the elongate shaft of the intravascular device shown in FIG. 1;

FIG. 2B is a cross-sectional view and FIG. 3B is a partial isometric view of another embodiment of the elongate shaft of the intravascular device shown in FIG. 1;

FIG. 2C is a cross-sectional view of a further embodiment of the elongate shaft of the intravascular device shown in FIG. 1; and

FIG. 2D is a cross-sectional view of yet another embodiment of the elongate shaft of the intravascular device shown in FIG. 1.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

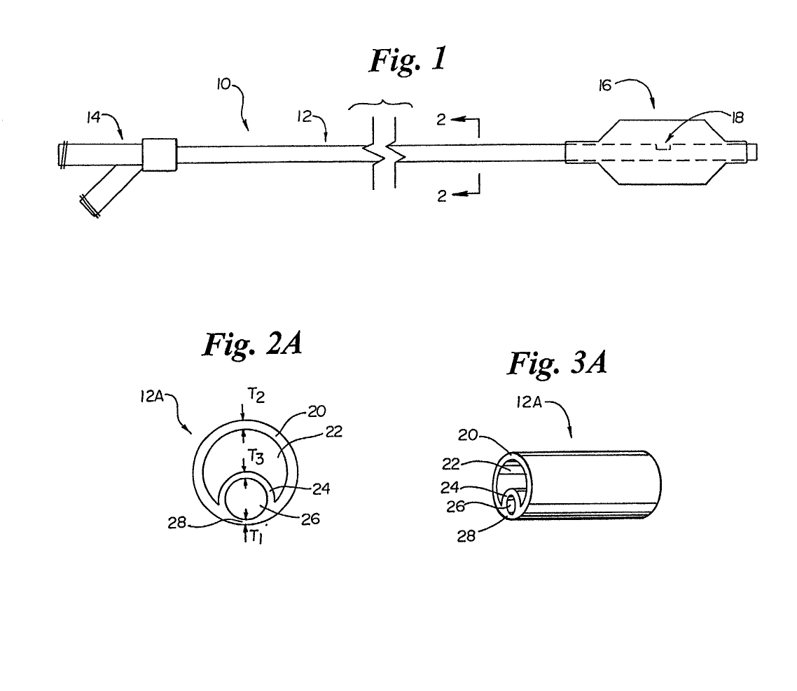

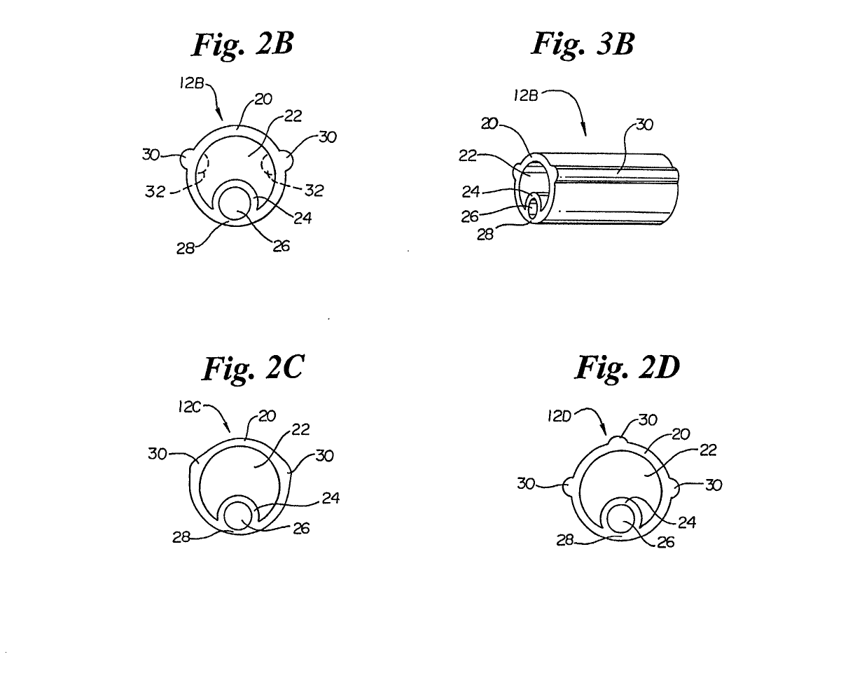

Refer now to FIG. 1, which illustrates a plan view of an intravascular device in the form of a balloon catheter 10. Those skilled in the art will recognize that the present invention may be implemented in a wide variety of intravascular devices, such as infusion catheters, guide catheters, diagnostic catheters, atherectomy devices and balloon catheters such as balloon catheter 10. Balloon catheter 10 includes an elongate shaft 12 having a proximal end and a distal end. A conventional manifold 14 is connected to the proximal end of the elongate shaft 12. Manifold 14 facilitates connection to an inflation device to inflate and deflate a balloon 16 mounted to the distal end of the elongate shaft 12. Fluid communication between the manifold 14 and the inflatable balloon 16 is provided by way of an inflation lumen 22 (visible in FIG. 2A) and an inflation port 18. Manifold 14 also facilitates insertion of a guidewire (not shown) into the guidewire lumen 26 (visible in FIG. 2A) which extends to the distal end of the elongate shaft 12. With the exception of the elongate shaft 12 and its features discussed hereinafter, intravascular balloon catheter 10 is substantially conventional. FIGS. 2A-2D describe various embodiments (12A,12B,12C,12D) of the elongate shaft 12 of the intravascular balloon catheter 10 illustrated in FIG. 1.

Refer now to FIG. 2A, which illustrates a cross-sectional view of a first embodiment of the elongate shaft 12A taken along line 2-2 in FIG. 1. Also refer to FIG. 3A, which illustrates an isometric view of a segment of the elongate shaft 12A. In this particular embodiment, the elongate shaft 12A includes an outer wall 20 and an inner wall 24. As used herein for purposes of description, the outer wall 20 refers to the entire wall defining the circumference of the elongate shaft 12A, and the inner wall 24 refers to the wall segment extending between two points inside the outer wall 20.

The outer wall 20 defines the majority of the inflation lumen 22. The inner wall 24 and a portion of the outer wall 20 define the guidewire lumen 26. In this particular example, the inflation lumen 22 is crescent-shaped and larger than the circle-shaped guidewire lumen 26. Those skilled in the art will recognize that that size, shape and position of the inner wall 24 may be varied to change the size, shape and geometry of the inflation lumen 22 and the guidewire lumen 26. In addition, those skilled in the art will recognize that the lumens 22,26 may be varied in number and function depending on the particular intravascular device implementing the concepts of the present invention.

As seen in FIG. 2A, the portion of the outer wall 20 which defines the guidewire lumen 26 includes a thinned portion 28 extending longitudinally along the shaft 12A. The thinned portion 28 of the outer wall 20 has a wall thickness T₁, which is less than the wall thickness T₂ of the remainder of the outer wall 20. The thickness T₁, of the thinned portion 28 may also be less than the wall thickness T₃ of the inner wall 24. The reduced wall thickness T₁ of the thinned portion 28 compensates for the imbalance of material and flexibility relative to the center longitudinal axis of the elongate shaft 12A due to the inner wall 24. In FIG. 2A, the center longitudinal axis of the elongate shaft 12A appears as a point (not shown) positioned at the geometric center of the outer wall 20. The provision of the inner wall 24 increases the amount of material on one side of the shaft 12A when viewed in cross section. The increased amount of material due to the inner wall 24 increases the rigidity along that side of the elongate shaft 12A, thereby causing non-uniformity in flexibility in different planes of flexure. By reducing the wall thickness T₁, in the thinned outer wall portion 28, the imbalance of material and flexibility due to the inner wall 24 is mitigated.

Because the thinned portion 28 of the outer wall 20 does not define any portion of the inflation lumen 22, the thinned portion 28 does not compromise the ability of the inflation lumen 22 to withstand high inflation pressures. In addition, the inner wall 24 may be shifted toward the thinned portion 28 of the outer wall 20 a distance approximately equal to T₂−T₁ without compromising the size of the guidewire lumen 26. Because the inner wall 24 may be shifted in the direction of the thinned portion 28 of the outer wall 20, the inflation lumen 22 also benefits from a corresponding increase in cross-sectional area, thereby improving fluid flow therethrough.

Refer now to FIG. 2B, which illustrates a cross-sectional view of an elongate shaft 12B in accordance with another embodiment of the present invention. Also refer to FIG. 3B, which illustrates an isometric view of a segment of the elongate shaft 12B. Except as illustrated and described herein, the elongate shaft 12B is substantially the same as elongate shaft 12A described with reference to FIGS. 2A and 3A.

Elongate shaft 12B includes an outer wall 20, an inner wall 24, an inflation lumen 22 and a guidewire lumen 26. Elongate shaft 12B may optionally include a thinned region 28 in the outer wall 20. Elongate shaft 12B further includes longitudinally extending spines 30 to further compensate for the imbalance of material and flexibility about the center longitudinal axis of the shaft 12B that would otherwise occur due to the inner wall 24. Relative to the center longitudinal axis, the longitudinal spines 30 are disposed on the opposite side of the inner wall 24 and the thinned portion 28 of the outer wall 20.

The longitudinal spines 30 may comprise discrete components connected to the outer wall 20. Alternatively, the longitudinal spines 30 may comprise integral components of the outer wall 20 such as an increase in thickness of the outer wall 20. Preferably, the longitudinal spines 30 are integrally formed with the outer wall 20 during extrusion. The longitudinal spines 30 may extend outwardly from the outer wall 20 (as shown) to maintain the size of the inflation lumen 22. Alternatively, the longitudinal spines 32 (shown in phantom) may extend inwardly into the inflation lumen 22 to maintain the outside profile of the elongate shaft 12B.

The longitudinal spines 30 may be positioned, relative to the center longitudinal axis of the elongate shaft 12B, opposite the inner wall 24 and the thinned portion 28 of the outer wall 20. The longitudinal spines 30 may be positioned equidistant from the inner wall 24 and/or thinned portion 28 of the outer wall 20. Preferably, the longitudinal spines 30 are uniformly spaced along the outer wall 20 opposite the inner wall 24 and thinned portion 28 of the outer wall 20 to increase the balance of material and flexibility about the center axis of the elongate shaft 12B.

Those skilled in the art will recognize that the size, shape and number of longitudinal spines 30 may be varied depending on the size, shape and position of the inner wall 24. For example, it is contemplated that a single longitudinal spine 30 may be positioned immediately opposite the inner wall 24 and thinned portion 28 of the outer wall 20. If two longitudinal spines 30 are utilized (as shown), the spines 30 may be positioned approximately one-third the radius of the outer wall 20 from the longitudinal center axis of the elongate shaft 12B to properly counterbalance the material of the inner wall 24.

As mentioned previously, the size, shape and number of longitudinal spines 30 may be varied depending on the degree of counterbalance needed to balance the material and flexibility of the elongate shaft 12. FIGS. 2C and 2D illustrate examples of variations in the size, number and position of the longitudinal spines 30. FIG. 2C illustrates elongate shaft 12C having two longitudinal spines 30 with a smoother outside surface than elongate shaft 12B. FIG. 2D illustrates an elongate shaft 12D having three longitudinal spines 30 uniformly spaced about the outer wall 20 opposite the inner wall 24 relative to the longitudinal center axis.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

What is claimed is:

1. An intravascular device comprising an elongate tubular shaft having an outer wall and an inner wall dividing the outer wall into first and second longitudinal lumens, wherein a portion of the outer wall has a reduced wall thickness to compensate for an imbalance of material and flexibility about a longitudinal center axis of the shaft that would otherwise occur due to the inner wall.

2. An intravascular device as in claim 1, wherein the first lumen is larger than the second lumen.

3. An intravascular device as in claim 2, wherein the first lumen is crescent shaped.

4. An intravascular device as in claim 3, wherein the second lumen is circular.

5. An intravascular device as in claim 1, further comprising one or more spines extending longitudinally along the outer wall to further reduce the imbalance of material and flexibility about the longitudinal center axis of the shaft that would otherwise occur absent the spines.

6. An intravascular device as in claim 5, wherein the one or more spines are integral with the outer wall.

7. An intravascular device as in claim 6, wherein the one or more spines comprise regions of increased wall thickness in the outer wall.

8. An intravascular device as in claim 7, wherein the one or more spines extend outwardly.

9. An intravascular device as in claim 7, wherein the one or more spines extend inwardly.

10. An intravascular device as in claim 5, wherein the one or more spines are positioned, relative to the center longitudinal axis, opposite the inner wall.

11. An intravascular device as in claim 10, wherein the one or more spines are positioned equidistant from the inner wall.

12. An intravascular device as in claim 10, wherein the one or more spines are uniformly spaced.

13. An intravascular device comprising an elongate tubular shaft having an outer wall and an inner wall dividing the outer wall into first and second longitudinal lumens, and one or more spines extending longitudinally along the outer wall to compensate for an imbalance of material and flexibility about a longitudinal center axis of the shaft that would otherwise occur due to the inner wall.

14. An intravascular device as in claim 13, wherein the first lumen is larger than the second lumen.

15. An intravascular device as in claim 14, wherein the first lumen is crescent shaped.

16. An intravascular device as in claim 15, wherein the second lumen is circular.

17. An intravascular device as in claim 13, wherein the spines are integral with the outer wall.

18. An intravascular device as in claim 17, wherein the spines comprise regions of increased wall thickness in the outer wall.

19. An intravascular device as in claim 18, wherein the spines extend outwardly.

20. An intravascular device as in claim 18, wherein the spines extend inwardly.

21. An intravascular device as in claim 13, wherein a portion of the outer wall defining the second smaller lumen has a reduced wall thickness to further reduce the imbalance of material and flexibility about the longitudinal center axis of the shaft that would otherwise occur.

22. An intravascular device comprising an elongate tubular shaft having an outer wall and an inner wall dividing the outer wall into first and second longitudinal lumens, and one or more regions of modified flexibility extending longitudinally along the outer wall to reduce an imbalance of material and flexibility about a longitudinal center axis of the shaft that would otherwise occur absent the regions of modified flexibility.

23. An intravascular device as in claim 22, wherein the first lumen is larger than the second lumen.

24. An intravascular device as in claim 23, wherein the first lumen is crescent shaped.

25. An intravascular device as in claim 24, wherein the second lumen is circular.

26. An intravascular device as in claim 22, wherein the one or more regions of modified flexibility comprise one or more regions of decreased wall thickness in the outer wall.

27. An intravascular device as in claim 22, wherein the one or more regions of modified flexibility comprise one or more spines extending longitudinally along the outer wall.

28. An intravascular device as in claim 27, wherein the spines are integral with the outer wall.

29. An intravascular device as in claim 28, wherein the spines comprise regions of increased wall thickness in the outer wall.

30. An intravascular device as in claim 29, wherein the spines extend outwardly from the outer wall.

31. An intravascular device as in claim 22, wherein the one or more regions of modified flexibility comprise a combination of one or more regions of decreased wall thickness in the outer wall and one or more spines extending longitudinally along the outer wall.

32. An intravascular device as in claim 31, wherein the one or more spines are positioned, relative to the center longitudinal axis, opposite the inner wall.

33. An intravascular device as in claim 32, wherein the one or more spines are positioned equidistant from the inner wall.

34. An intravascular device as in claim 32, wherein the one or more spines are uniformly spaced.
